(19) United States
(12) Patent Application Publication
(10) Pub. No.: US 2026/0022334 A1
(43) Pub. Date: Jan. 22, 2026

(54) COMPOSITION, METHOD, AND APPLICATION THEREOF FOR IMPROVING THE QUALITY OF IN VITRO MATURATION OF OOCYTES AND PREGNANCY RATE OF EMBRYOS AFTER IN VITRO FERTILIZATION

(71) Applicant: CHINA AGRICULTURAL UNIVERSITY, Beijing (CN)

(72) Inventors: Jianhui TIAN, Beijing City (CN); Guangyin XI, Beijing City (CN); Jia HAO, Beijing City (CN); Yuan YUE, Beijing City (CN); Shumin WANG, Beijing City (CN); Lei AN, Beijing City (CN); Min GUO, Beijing City (CN)

(21) Appl. No.: 19/057,132

(22) Filed: Feb. 19, 2025

(30) Foreign Application Priority Data
Jul. 17, 2024 (CN) 202410955964.5

(51) Int. Cl. C12N 5/073; C12N 5/00; C12N 5/075

(52) U.S. Cl. CPC C12N 5/0604; C12N 5/0018; C12N 5/0609; C12N 2500/33; C12N 2500/38; C12N 2501/20

(57) ABSTRACT

The present disclosure discloses a composition, method and application thereof for improving the quality of in vitro maturation of oocytes and the pregnancy rate after in vitro produced embryos transfer. The present disclosure aims at the problems of low maturation rate and poor subsequent embryo development potential caused by metabolic disorders during in vitro maturation of oocytes, and provides a composition including two or more regulators in vitamin C, L-carnitine and thrombopoietin to regulate the metabolism of oocytes, thereby greatly improving the in vitro maturation rate of oocytes and the subsequent embryo development potential. The present disclosure not only improves the efficiency of in vitro embryo production of livestock, but also provides a reference for improving the efficiency of human assisted reproductive technology. The method is simple, safe and efficient, and has great application value and broad application prospects.

### TECHNICAL FIELD

The present disclosure belongs to the field of biotechnology, and particularly relates to a composition, method and application thereof for improving in vitro maturation quality of oocytes and the pregnancy rate of embryos after in vitro fertilization by the combined administration of vitamin C, L-carnitine, and THPO.

### BACKGROUND

In the past decades, the in vitro mammalian embryos production technology has been widely used in fields of livestock genetic improvement, elite breed propagation, and human assisted reproduction. However, in vitro embryos face technical bottlenecks such as low development efficiency and poor embryo quality, which have been difficult to overcome. At present, it is widely believed that the difference between the in vitro culture environment and the in vivo development environment, particularly the incomplete composition of the in vitro oocyte maturation culture medium leading to insufficient oocyte in vitro maturation, is an important cause of the low in vitro embryo development potential, particularly the low pregnancy rate after embryo transfer, which greatly limits the development of the in vitro embryo production technology. Therefore, optimizing the composition of the in vitro maturation medium to improve oocyte maturation quality, and then improving the subsequent embryo development potential is currently the most direct and effective strategy to improve the efficiency and quality of in vitro embryo development and improve in vitro embryo production technology.

Oocyte maturation is strictly modulated by various signals. Oocytes gradually develop to maturity in vivo as follicles develop. The follicle provides good material and environmental guarantees for the high-quality oocyte maturation. However, the in vitro oocyte maturation environment is an artificial culture medium, and the oxygen concentration in the culture environment is higher than that in the follicles in vivo, which can easily cause oxidative stress in the oocytes and cannot meet the high-quality maturation requirements of the oocytes. Furthermore, metabolism in the oocyte, especially lipid metabolism, provides sufficient energy for oocyte maturation. Under the in vivo environment, a complex and dynamic lipid metabolism regulation network is formed in the oocyte, but the key factors or pathways which play a central role are still unknown. Although lipid metabolites are indispensable, prolonged exposure to high lipid environment can also cause irreversible damage to oocytes.

At present, numerous strategies have been reported to improve the oocyte quality by adding antioxidants or promoting lipid metabolism substances to the in vitro maturation culture medium. For example, adding cysteamine to the in vitro maturation medium can increase the glutathione content in oocytes and reduce oxidative stress damage. Resveratrol, quercetin, and melatonin can reduce the level of reactive oxygen species in oocytes and the level of apoptosis in early embryos, and increase the number of mitochondria and ATP content in oocytes. CN100432219A discloses a bovine oocyte in vitro maturation culture medium containing the antioxidant tea polyphenol for eliminating free radicals such as hydroxyl radicals and superoxide anions, thereby improving the in vitro maturation rate of bovine oocytes. CN110846272A discloses a method for reducing the reactive oxygen species content in oocytes, which is obtained by adding 5-20% mature follicular fluid to the basal culture medium to improve the in vitro maturation rate of porcine oocytes.

However, due to the complexity of oxidative stress and lipid metabolism regulation, the current corrective technologies for oocyte in vitro maturation still has the problems of low development efficiency and poor quality.

The information provided in BACKGROUND is illustrative only of the general background of the present disclosure and should not be construed as an acknowledgment or in any way imply that this information forms the prior art, which is well known to those of ordinary skill in the art.

### SUMMARY

The present disclosure is based on the results of previous studies, which reveal that the thrombopoietin (THPO) gene is highly expressed in the mural granulosa cells of cattle and sheep. The major functional protein encoded by the THPO gene is a humoral growth factor, which is essential for megakaryocyte proliferation, maturation and platelet production, and can affect the metabolism of mitochondria in hematopoietic stem cells. Based on the aforementioned studies, the present disclosure creatively developed a composition for in vitro maturation culture of oocytes and established a novel method for in vitro maturation of oocytes. Specifically, the present disclosure provides a composition and application for improving the quality of in vitro maturation of oocytes and the pregnancy rate of embryos after in vitro fertilization.

A first aspect of the present disclosure provides a composition for improving the quality of in vitro maturation of oocytes and the pregnancy rate of embryos after in vitro fertilization, comprising two or more modulators selected from vitamin C or its precursor thereof, L-carnitine, and THPO.

In certain embodiments, in the composition for improving the quality of in vitro maturation of oocytes and the pregnancy rate of embryos after in vitro fertilization according to the present disclosure, the composition comprises any of combinations (a)-(d):
(a) Vitamin C or a precursor thereof and L-carnitine, with a weight ratio of vitamin C or the precursor thereof to L-carnitine of 1:2-50;
(b) Vitamin C or a precursor thereof and THPO, with a weight ratio of vitamin C or the precursor thereof to THPO of 200-1000:1;
(c) L-carnitine and THPO, with a weight ratio of L-carnitine to THPO of 1000-10000:1;
(d) Vitamin C or a precursor thereof, L-carnitine, and THPO, with a weight ratio of vitamin C or the precursor thereof to L-carnitine to THPO of (200-1000):(1000-10000):1.

In certain embodiments, in the composition for improving the quality of in vitro maturation of oocytes and the pregnancy rate of embryos after in vitro fertilization according to the present disclosure, the amount of vitamin C or its precursor can make the working concentration of vitamin C reach 0.5-500 μg/ml during in vitro culture; the amount of L-carnitine can make the working concentration of L-carnitine reach 10-1000 μg/ml during in vitro culture; or the amount of THPO can make the working concentration of THPO reach 0.001-5 μg/ml during in vitro culture.

In certain embodiments, the composition for improving the quality of in vitro maturation of oocytes and the pregnancy rate of embryos after in vitro fertilization according to the present disclosure further comprises a basal culture medium.

The second aspect of the present disclosure provides is a method for improving the quality of in vitro maturation of oocytes and the pregnancy rate of embryos after in vitro fertilization, which comprises the step of contacting the oocytes with the composition according to the first aspect in vitro.

In certain embodiments, in the method for improving the quality of in vitro maturation of oocytes and the pregnancy rate of embryos after in vitro fertilization according to the present disclosure, the improvement comprises improving development efficiency and/or quality of the embryo.

The third aspect of the present disclosure provides a method for improving in vitro maturation of oocytes, comprising the step of contacting the oocyte with the composition of the first aspect in vitro.

In certain embodiments, according to the method for improving in vitro maturation of oocytes described in the present disclosure, the improvement comprises increasing the blastocyst development rate, the number of blastocyst cells, reducing the number of blastocyst apoptotic cells, and increasing the pregnancy rate after embryo transfer.

The fourth aspect of the present disclosure provides use of the composition described in the first aspect in animal genetic improvement, elite breed propagation, and in vitro mammalian embryo production or culture.

According to the data released by the International Embryo Technology Society (IETS), the number of in vitro embryos of bovine has exceeded the number of in vivo embryos since 2016, and it has shown a tend of increasing year by year, making the in vitro embryo production technology an effective way to efficiently utilize elite breed resources. The present disclosure provides effective correction approaches and methods for the problems of low embryo development rate and low subsequent development potential caused by poor quality of egg in vitro maturation, greatly improves the development rate and quality of embryos after in vitro fertilization, and provides an effective strategy for the upgrading of the animal husbandry industry. Additionally, the present disclosure has significant reference value in the field of human assisted reproduction, especially in the acquisition of high-quality embryos. Therefore, the present disclosure will produce huge economic value and social value in the field of in vitro mammalian embryo production.

### BRIEF DESCRIPTION OF THE DRAWINGS

In order to more clearly illustrate the technical solutions in the embodiments or methods of the present disclosure, the drawings used in the embodiments are briefly introduced below. Obviously, the drawings in the following description are only some embodiments of the present disclosure. For those of ordinary technicians in this field, other drawings can be obtained according to these drawings without creative effort.

FIG. 1 Dynamic analysis of THPO gene expression of in mural granulosa cells and oocytes of follicles at different development stages in cattle and sheep. Among them, small (diameter<5 mm), medium (5 mm<diameter<12 mm), and large (diameter>12 mm) bovine follicles were collected from ovaries in slaughterhouse. Ovine follicles were collected before the first injection of FSH (FSH 0 h), 24 h after the first injection of FSH (FSH 24 h), 24 h after the second injection of FSH (FSH 48 h), before LH injection (LH 0 h), 8 h after the LH injection (LH 8 h), and 26 h after the LH injection (LH 26 h). Single-cell transcriptome sequencing was performed on mural granulosa cells and oocytes at each stage, and the results were analyzed to obtain TPM values.

FIG. 2 shows a flow diagram of oocyte in vitro maturation-in vitro fertilization-embryo in vitro culture-embryo transfer.

FIG. 3 Statistical analysis of the total number of blastocyst cells after different treatments of sheep oocyte in vitro maturation. Among them, the control group is a routine in vitro maturation group, and treatment groups 1-7 are: VitC alone group, L-carnitine alone group, THPO alone group, VitC and L-carnitine combined administration group, VitC and THPO combined administration group, L-carnitine and THPO combined administration group, VitC, L-carnitine, and THPO combined administration group. The total number of blastocyst cells were obtained by counting cells after DAPI immunofluorescence staining.

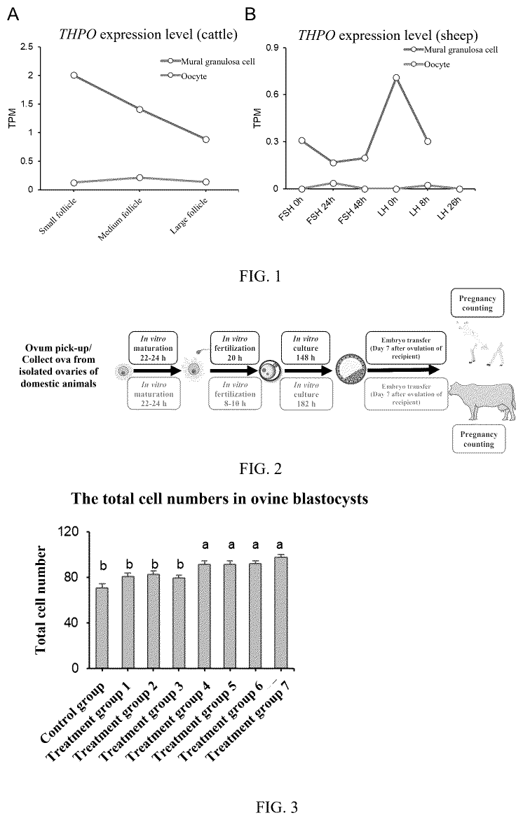

### DETAILED DESCRIPTION

Various exemplary embodiments of the present disclosure are described in detail below. The detailed description should not be construed as limitations on the present disclosure but as a more detailed description of certain aspects, features, and embodiments of the present disclosure.

It will be appreciated that the terms used herein are for the purpose of illustrating particular embodiments only, rather than limiting the present disclosure. In addition, for the numerical ranges in the present disclosure, it will be appreciated that the upper and lower limits of the ranges and each intermediate value therebetween are specifically disclosed. Every smaller range between any stated value or intervening value in the stated range and any other stated or intervening value in that stated range is encompassed within the present disclosure. The upper and lower limits of these smaller ranges can be independently included or excluded in the range.

Unless defined otherwise, all technical and scientific terms used herein have the same meaning as commonly understood by those skilled in the art to which the present disclosure pertains. Although only preferred methods and materials are described herein, any methods and materials similar or equivalent to those described herein can be used in the practice or testing of the present disclosure. All documents described herein are incorporated by reference to disclose and describe the methods and/or materials associated with the documents. In case of conflict with any incorporated document, the specification shall prevail.

As used herein, the term “oocyte” refers to a cumulus-oocyte complex obtained from the oocyte pick-up procedure or extracted from isolated ovarian follicles.

As used herein, the term “embryo” refers to an early embryo developed from a zygote. Since development is a continuous process, embryos herein include preimplantation embryos, such as 2-cell embryos, 4-cell embryos, 8-cell embryos, morulae, and blastocysts.

As used herein, the term “in vitro” describes an event that occurs in an artificial environment, for example, in a test tube or reaction vessel, in cell culture, or in a culture dish, rather than in an organism (e.g., animal, plant, or microorganism).

As used herein, the term “modulate”, “modulating”, “modulated”, or “modulation”, which may sometimes also be referred to as “regulate”, “regulating”, “regulated”, or “regulation”, refers to any action that alters the level of oxidative stress or lipid metabolism.

As used herein, the term “vitamin C” or “ascorbic acid”, abbreviated as “VitC”, is associated with a variety of diseases and is determined by the World Health Organization as an essential medicine in the list of essential medicines.

As used herein, the term “precursor” refers to any substance, molecule, or entity which is capable of acting as or producing vitamin C after being chemically or physically altered. The precursor may be covalently bound or chelated in certain manner, and release or convert into an active ingredient, particularly vitamin C. The precursor may be prepared by modifying a functional group present in the compound in such a way that the modified compound can be cleaved to release the parent compound in routine manipulation or in vivo. The precursor includes compounds in which a hydroxyl, amino, sulfhydryl, or carboxyl group is bonded to any group that is cleaved to form a free hydroxyl, amino, sulfhydryl, or carboxyl group, respectively, when administered to a mammalian subject.

As used herein, the term “L-carnitine”, also referred to as levocarnitine or vitamin BT, with the chemical formula C₇H₁₅NO₃, is an amino acid analog that promotes the conversion of fat into energy.

As used herein, the term “THPO”, the full name of which is Thrombopoietin, is closely related to the metabolic regulation of hematopoietic stem cells.

As used herein, the term “working concentration” is also referred to as “use concentration”, which refers to an initial concentration at which a modulator is brought into contact with the oocyte for effective treatment. The concentration unit is not limited and may be, for example, ng/ml, μg/mL, or mg/mL.

### Composition

In a first aspect of the present disclosure, there is provided a composition for improving the quality of in vitro maturation of oocytes and the pregnancy rate of embryos after in vitro fertilization. The composition is sometimes referred to herein as “composition of the present disclosure” and comprises a combination of different modulators, wherein the modulators are selected from two or more of vitamin C or a precursor thereof, L-carnitine, and THPO. The modulators in the combination of the present disclosure interact with each other to produce a synergistic effect. Compared with a single modulator, the modulator combination has a greatly improved regulatory effect. The composition of the present disclosure is not limited to a form and may be in the form of a solid such as a dry powder or a liquid such as a medium.

In certain embodiments, the composition of the present disclosure may be a combination of the modulators described above; that is, the composition comprises no ingredients other than the modulators and unavoidable impurities. The modulators may be present in a mixed form in the composition of the present disclosure, or two or more modulators may be present separately. When in use, the modulators which are present separately may be pre-mixed before use, or the modulators may be used separately and simultaneously, or separately and sequentially.

In certain embodiments, the composition of the present disclosure comprises the modulator combination described above and further comprises other ingredients. The composition or type of the other ingredients is not limited and may be freely selected as required. Such other ingredients may be selected from any known ingredients, in particular reagents and compositions related to embryo culture, such as culture mediums or additives.

In certain embodiments, the modulator combination of the present disclosure is a combination of vitamin C or a precursor thereof and L-carnitine. Preferably, the weight ratio of vitamin C to L-carnitine is 1:2-50, for example, 1:2, 1:5, 1:10, 1:15, 1:20, 1:25, 1:30, 1:35, 1:40, 1:45, or 1:50. Within the range described above, an effective synergy can be achieved between vitamin C and L-carnitine, significantly improving the regulatory effect.

In certain embodiments, the modulator combination of the present disclosure is a combination of vitamin C or a precursor thereof and THPO. Preferably, the weight ratio of vitamin C to THPO is 200-1000:1, for example, 200:1, 250:1, 300:1, 350:1, 400:1, 450:1, 500:1, 550:1, 600:1, 650:1, 700:1, 750:1, 800:1, 850:1, 900:1, 950:1, or 1000:1. Within the range described above, an effective synergy can be achieved between vitamin C and THPO, significantly improving the regulatory effect.

In certain embodiments, the modulator combination of the present disclosure is a combination of L-carnitine and THPO. Preferably, the weight ratio of L-carnitine to THPO is 1000-10,000:1, for example, 1000:1, 1200:1, 1400:1, 1600:1, 1800:1, 2000:1, 2200:1, 2400:1, 2500:1, 2750:1, 3000:1, 3500:1, 4000:1, 4500:1, 5000:1, 5500:1, 6000:1, 6500:1, 7000:1, 7500:1, 8000:1, 8500:1, 9000:1, 9500:1, or 10,000:1. Within the range described above, an effective synergy can be achieved between L-carnitine and THPO, significantly improving the regulatory effect.

In certain embodiments, vitamin C or the precursor thereof of the present disclosure is present in an amount capable of achieving a working concentration of 0.5-500 μg/mL when in use. The concentration or amount of vitamin C in the composition is not limited as long as it can achieve the range described above when vitamin C is in use. When the composition is a solution and serves as an additive ingredient, the concentration of vitamin C or the precursor thereof in the composition can be higher than the working concentration, so that when the composition is added to the embryo culture medium, it can be diluted to achieve the above working concentration. In the case that the composition is a solution and is used directly as a culture medium, the concentration of vitamin C or the precursor thereof in the composition is usually substantially equal to the working concentration. The working concentration of vitamin C is preferably 1-400 μg/mL, such as 1.5 μg/mL, 2 μg/mL, 2.5 μg/mL, 3 μg/mL, 3.5 μg/mL, 4 μg/mL, 4.5 μg/mL, 5 μg/mL, 5.5 μg/mL, 6 μg/mL, 6.5 μg/mL, 7 μg/mL, 7.5 μg/mL, 8 μg/mL, 8.5 μg/mL, 9 μg/mL, 9.5 μg/mL, 10 μg/mL, 11 μg/mL, 12 μg/mL, 13 μg/mL, 14 μg/mL, 15 μg/mL, 16 μg/mL, 17 μg/mL, 18 μg/mL, 19 μg/mL, 20 μg/mL, 21 μg/mL, 22 μg/mL, 25 μg/mL, 30 μg/mL, 35 μg/mL, 40 μg/mL, 50 μg/mL, 60 μg/mL, 70 μg/mL, 80 μg/mL, 90 μg/mL, 100 μg/mL, 120 μg/mL, 140 μg/mL, 160 μg/mL, 180 μg/mL, 200 μg/mL, 220 μg/mL, 240 μg/mL, 260 μg/mL, 280 μg/mL, 300 μg/mL, 350 μg/mL, or 400 μg/mL.

In certain embodiments, the amount of L-carnitine of the present disclosure can make the working concentration of L-carnitine reach 10-1000 g/mL. The concentration or amount of L-carnitine in the composition is not limited as long as it can achieve the range described above when L-carnitine is in use. In the case where the composition is a solution and serves as an additive ingredient, the concentration of L-carnitine in the composition can be higher than the working concentration, so that when the composition is added to the embryo culture medium, it can be diluted to achieve the above working concentration. In the case that the composition is a solution and is used directly as a culture medium, the concentration of L-carnitine in the composition is usually substantially equal to the working concentration. The working concentration of L-carnitine is preferably 50-500 μg/mL, such as 55 μg/mL, 60 μg/mL, 65 μg/mL, 70 μg/mL, 75 μg/mL, 80 μg/mL, 85 μg/mL, 90 μg/mL, 95 μg/mL, 100 μg/mL, 125 μg/mL, 150 μg/mL, 175 μg/mL, 200 μg/mL, 225 μg/mL, 250 μg/mL, 275 μg/mL, 300 μg/mL, 325 μg/mL, 350 μg/mL, 375 μg/mL, 400 μg/mL, 425 μg/mL, 450 μg/mL, 475 μg/mL, or 500 μg/mL.

In certain embodiments, the amount of THPO of the present disclosure can make the working concentration of THPO reach 0.001-5 μg/mL. The working concentration of THPO is preferably 0.05-3 μg/mL, such as 0.06 μg/mL, 0.07 μg/mL, 0.08 μg/mL, 0.09 μg/mL, 0.1 μg/mL, 0.15 μg/mL, 0.20 μg/mL, 0.25 μg/mL, 0.3 μg/mL, 0.35 μg/mL, 0.4 μg/mL, 0.45 μg/mL, 0.5 μg/mL, 0.55 μg/mL, 0.6 μg/mL, 0.65 μg/mL, 0.7 μg/mL, 0.75 μg/mL, 0.8 μg/mL, 0.85 μg/mL, 0.9 μg/mL, 0.95 μg/mL, 1 μg/mL, 1.5 μg/mL, 2 μg/mL, 2.5 μg/mL, or 3 μg/mL.

### Methods for Improving In Vitro Maturation of Oocytes

The second aspect of the present disclosure provides a method for improving in vitro maturation of oocytes, comprising the step of contacting the oocytes with the composition of the first aspect in vitro.

The improvements of the present disclosure include increasing embryo development efficiency and/or quality, improving the blastocyst development rate, the number of blastocyst cells, and the implantation rate after embryos transfer.

### Use

The third aspect of the present disclosure provides the use of the composition of the first aspect in animal genetic improvement, elite breed propagation, and in vitro mammalian embryo production. The composition and the modulators thereof have been described in detail in the first aspect and will not be described here again.

In certain embodiments, the animal of the present disclosure refers to a mammal, for example, mouse, rat, rabbit, monkey, dog, cat, sheep, cow, pig, primate. In some embodiments, the animal includes but not limited to mammals, birds, reptiles, amphibians, fish, and worms. In some embodiments, the animal is a transgenic animal, a genetically engineered animal, or a cloned animal. In certain embodiments, the animal of the present disclosure is a domestic animal, including pigs, cows, and sheep.

In certain embodiments, the genetic improvement and elite breed propagation of the present disclosure include genetic improvement or propagation that is dependent on the in vitro embryo production technology.

### Example 1

Use of Combined Administration of VitC, L-Carnitine, and THPO in Improvement of in Vitro Maturation Quality and Development Potential of Ovine oocytes

### 1. Experimental Material

### Collection of Ovine Oocytes (Cumulus-Oocyte Complexes, COCs)

Ovine ovaries were collected in a slaughterhouse, placed in normal saline at 35° C., and transported to the laboratory within 2 h. The ovaries were washed three times with normal saline, and follicles were punctured using a 20 G needle containing 5 mL of oocyte collection medium to release cumulus-oocyte complexes. The COCs with uniform cytoplasm and at least 3 layers of cumulus cells were collected Under a stereomicroscope. The subsequent experimental procedures are shown in FIG. 2.

### 2. Experimental Method

### (1) Grouping:

Control group: a routine in vitro maturation group.
Treatment group 1: a VitC (100 μg/mL)—only administration group.
Treatment group 2: an L-carnitine (1 mg/mL)—only administration group.
Treatment group 3: a THPO (200 ng/mL)—only administration group.
Treatment group 4: a VitC (50 μg/mL) and L-carnitine (500 μg/mL) combined administration group.
Treatment group 5: a VitC (50 μg/mL) and THPO (100 ng/ml) combined administration group.
Treatment group 6: an L-carnitine (500 μg/mL) and THPO (100 ng/ml) combined administration group.
Treatment group 7: a VitC (33.3 μg/mL), L-carnitine (333.3 μg/mL), and THPO (66.7 ng/mL) combined administration group.

The control group was a basal in vitro maturation medium, and the treatment groups (1-7) were prepared by adding different components or combined components to the basal maturation medium.

### (2) In Vitro Maturation of Ovine Oocytes

The collected ovine oocytes were washed three times with washing medium, then washed 3 times with the basal in vitro oocyte maturation medium pre-equilibrated for 3 h in advance, transferred to the in vitro maturation culture mediums of the control and experimental groups, and incubated in a 5% CO₂, 38.5° C. incubator with saturated humidity for 22-24 h. Four-well plates were used for culture, and each well contained 600 μL of in vitro maturation culture medium and 30-35 ovine cumulus-oocyte complexes, and was covered with 300 μL of mineral oil. In vitro maturation of ovine oocytes was performed according to different groups.

For the control group, the basal in vitro oocyte maturation medium was used. The basal in vitro oocyte maturation medium was prepared as follows: TCM199+10% estrus ovine serum+1 μg/mL follicle-stimulating hormone+1 μg/mL luteinizing hormone+1 μg/mL β-estradiol+12 μg/mL DL-cysteine+100 μg/mL streptomycin+100 IU/mL penicillin.

### (3) In Vitro Fertilization of Ovine Oocytes

After in vitro maturation, the matured oocytes were subjected to in vitro fertilization (IVF). Briefly, COCs were washed three times in synthetic oviductal fluid (SOF) medium with 0.2% hyaluronidase to remove cumulus cells and transferred into the IVF medium, which consisted of synthetic oviduct fluid (SOF) medium containing 2% estrus sheep serum (ESS), 3 mg/mL BSA, 6 IU/mL heparin sodium and 50 IU/mL gentamicin. For IVF, frozen semen was removed from the liquid nitrogen and thawed in a water bath at 39° C. for 1 min. Then, the sperm diluted with IVF medium and swimming up for 30 min at 38.5° C. in a humidified atmosphere at 5% CO₂. The upper layer of semen was transferred into a 1.5 mL tube and then centrifuge for 5 min at 2000 rpm. The spermatozoa were carefully collected from the bottom of the centrifuge tube and added into each IVF medium with a final concentration of approximately 1×10⁶ sperm/mL. Then, the oocyte and sperm were co-incubated for 20 h at 38.5° C. with 5% CO₂ in a humidified atmosphere. All reagents were purchased from Sigma-Aldrich.

### (4) In Vitro Culture of Ovine Embryos

After IVF, presumptive zygotes were washed three times in IVC medium and then transferred to SOF medium supplemented with 1% BME-essential amino acids, 1% MEM-nonessential amino acids, 1 mM glutamine and 3 mg/mL BSA and cultured at 38.6° C. in a humidified environment with an 88% N₂, 5% CO₂, 7% O₂. The cleavage rate and blastocyst rate were recorded at 48 h and 6-7 d after IVF. After 48 h of culture, the cleavage rate was calculated. The culture was continued until day 7, and the blastocyst rate was calculated.

### (5) Count the Total Number of Blastocyst Cells

The 7d blastocysts were collected, washed 3 times with PBS containing 1 mg/ml PVA (PBS-PVA), and stained with DAPI, and the total blastocyst cell number was then determined. Specifically, the zona pellucida was removed with Tyrode's medium, and the blastocysts were then fixed in PBS containing 4% polyformaldehyde for 30 min and permeabilized using PBS containing 0.5% TritonX-100 for 20 min at room temperature. The nuclei of the blastocysts were stained with 1 μg/mL DAPI, incubated for 5 min in the dark, and then mounted onto slides. DAPI cells were observed and counted under a fluorescence microscope.

### (6) Ovine Embryo Transfer and Pregnancy Assessment

Transferrable embryo were selected and one blastocyst was transferred into recipient ewes using a laparoscopic procedure. Prior to embryo transfer, the ewes were fasted for at least 20 h. The embryos were transplanted into recipients with an assistant of endoscopic apparatus. The conception was detected at 45 d after embryo transfer by using B-ultrasonography and the embryo transfer pregnancy rate was calculated using the formula:

$$P regnancy rate = ( number of pregnant recipient sheep / total number of transfer recipient sheep ) \times 100 \% .$$

### 3. Test Results

Statistical results of in vitro embryo development rates are shown in Table 1, and total blastocyst cell counts are shown in FIG. 3.

The data in Table 1 shows that the cleavage rate of experimental group 7 (84.44±3.56%), in which vitamin C, L-carnitine, and THPO were added, was significantly (p<0.05) higher than that of the control group (71.16±2.19%). VitC+L-carnitine, VitC+THPO, L-carnitine+THPO, and VitC+L-carnitine+THPO produced synergistic effects and all achieved significantly higher blastocyst development efficiency than the VitC-only, L-carnitine-only, and THPO-only treatment groups, and the combination of VitC, L-carnitine, and THPO was the most effective of these combinations. It can be seen that the combined use of VitC, L-carnitine, and THPO helps to increase the development efficiency of in vitro fertilization ovine embryos.

TABLE 1
The effect of the combined administration of VitC, L-carnitine,
and THPO during the in vitro maturation on the embryo
development rate after in vitro fertilization.

| | Number of | | D7 blastocyst |
|---|---|---|---|
| Groups | oocytes | Cleavage rate (%) | rate (%) |
| Control group | 224 | 71.16 ± 2.19^{a} | 32.39 ± 1.69^{a} |
| Treatment group 1 | 233 | 75.64 ± 2.83^{a} | 38.79 ± 1.97^{b} |
| Treatment group 2 | 251 | 74.32 ± 2.31^{a} | 37.61 ± 1.85^{b} |
| Treatment group 3 | 232 | 75.38 ± 2.43^{a} | 38.01 ± 1.50^{b} |
| Treatment group 4 | 230 | 74.57 ± 2.65^{a} | 44.06 ± 2.02^{c} |
| Treatment group 5 | 225 | 73.39 ± 2.16^{a} | 46.37 ± 2.15^{c} |
| Treatment group 6 | 230 | 75.02 ± 2.64^{a} | 48.35 ± 2.74^{c} |
| Treatment group 7 | 228 | 84.44 ± 3.56^{b} | 50.82 ± 2.83^{c} |

Note:
The development rate data were obtained through one-way analysis of variance test, expressed as mean ± standard error. Different superscript letters within the same column, ^{a}, ^{b}, and ^{c}, indicate significant differences (p < 0.05).

The total cell number of blastocyst is an important indicator for evaluating the quality of blastocyst. The data in FIG. 3 shows that VitC+L-carnitine (91.0±3.0), VitC+THPO (91.4±2.6), L-carnitine+THPO (91.9±2.5), and VitC+L-carnitine+THPO (97.5±2.6) produced synergistic effects and all achieved significantly higher cell number than VitC-only (80.6±3.2), L-carnitine-only (82.6±2.7), and THPO-only (79.4±2.3) treatment groups, and the combination of VitC, L-carnitine, and THPO was the most effective of these combinations. It can be seen that the combined use of VitC, L-carnitine, and THPO can help increase the cell number of in vitro fertilized embryos.

The in vitro matured oocyte of each group were subjected to in vitro fertilization and embryo culture, and the embryos were transferred and the pregnancy rates were calculated. The results are shown in Table 2. The results showed that compared with the control group (50.9%), the combination treatments of VitC+L-carnitine, VitC+THPO, L-carnitine+THPO, and VitC+L-carnitine+THPO produced synergistic effects and all achieved significantly higher transfer efficiency than the VitC-only, L-carnitine-only, and THPO-only treatment groups, and the combination of VitC, L-carnitine, and THPO was the most effective (66.7%) of these combinations and was significantly more effective than the combinations of two of VitC, L-carnitine, and THPO. It can be seen that the combined use of VitC, L-carnitine, and THPO is the most effective scheme to improve the pregnancy rate after embryos transfer.

TABLE 2
The effect of the combined administration of VitC, L-
carnitine, and THPO during the in vitro maturation of
oocytes on the pregnancy rate after embryo transfer.

| | Number of | Number of | |
|---|---|---|---|
| Group | transfers | pregnancies | Pregnancy rate (%) |
| Control group | 53 | 27 | 50.9^{a} |
| Treatment group 1 | 50 | 25 | 50.0^{a} |
| Treatment group 2 | 48 | 26 | 54.2^{a} |
| Treatment group 3 | 49 | 26 | 53.1^{a} |
| Treatment group 4 | 45 | 27 | 60.0^{b} |
| Treatment group 5 | 43 | 26 | 60.5^{b} |
| Treatment group 6 | 48 | 29 | 62.5^{b} |
| Treatment group 7 | 51 | 34 | 66.7^{c} |

Note:
The pregnancy rate data were obtained through chi-square test analysis. Different superscript letters within the same column, ^{a}, ^{b}, and ^{c}, indicate significant differences between different groups (p < 0.05).

### Example 2

Use of Combined Administration of VitC, L-Carnitine, and THPO in Improving the In Vitro Maturation Quality and Development Potential of Bovine oocytes

### 1. Experimental Design and Method

### (1) Grouping

Control group: a routine in vitro maturation group;
Treatment group 1: a VitC (100 μg/mL)—only administration group;
Treatment group 2: an L-carnitine (1 mg/mL)—only administration group;
Treatment group 3: a THPO (200 ng/mL)—only administration group;
Treatment group 4: a VitC (50 μg/mL) and L-carnitine (500 μg/mL) combined administration group;
Treatment group 5: a VitC (50 μg/mL) and THPO (100 ng/ml) combined administration group;
Treatment group 6: an L-carnitine (500 μg/mL) and THPO (100 ng/mL) combined administration group;
Treatment group 7: a VitC (33.3 μg/mL), L-carnitine (333.3 μg/mL), and THPO (66.7 ng/mL) combined administration group.

The control group was a basal in vitro maturation medium, and the treatment groups (1-7) were prepared by adding different components or combined components to the basal maturation medium.

### (2) In Vitro Maturation of Bovine Oocytes and Embryo Culture

The experimental procedures are shown in FIG. 2.

### 1) Oocyte Collection, In Vitro Maturation

Bovine ovaries were collected from a local slaughterhouse (Dachang Hui Autonomous County, Hebei Province, China) and transported to the laboratory within two hours in saline solution containing penicillin and streptomycin. In the laboratory, bovine COCs were aspirated from follicles with a diameter of 2-8 mm to M199-HEPES containing 20 μg/mL heparin sodium, 44 μg/mL sodium pyruvate, 70 μg/mL penicillin and 50 μg/mL streptomycin. The aspirated follicular fluid was placed in 50 ml conical tubes and maintained for a period for sedimentation. The upper portion of the liquid was removed and the remaining portion was then transferred to wash buffer. COCs with a homogenous, evenly granulated ooplasm surrounded by at least three layers of compact cumulus cells were selected under a stereomicroscope for experiments. COCs were washed three times in BO-IVM medium and incubated for 22 h at 38.5° C. in a humidified atmosphere under 5% CO₂ in groups of 50 containing 500 μL BO-IVM medium in four-well dishes covered with mineral oil.

### 2) In Vitro Fertilization and In Vitro Embryo Culture

After 22 h of IVM, 20 COCs were transferred to 100 μL BO-IVF medium covered with mineral oil in Petri dish (60×15 mm). Frozen-thawed sperm was purified using BO-SemenPrep. Spermatozoa were selected by Percoll method and the final concentration was adjusted to 1×10⁶ in each drop. They were co-incubated in BO-IVF medium with humidified 5% CO₂ in air at 38.5° C. for 18 h. Thereafter, the presumptive zygotes were transferred to BO-IVC medium in four-well dishes covered with mineral oil, and incubated with humidified 5% CO₂ in air at 38.5° C. for 8 days. The cleavage rate was determined at 48 h and the blastocyst rate was estimated at 8d postinsemination.

### 3) Embryo Transfer and Pregnancy Rate Assessment.

The bovine recipients were restrained and anesthetized according to requirement of sartificial insemination. After the vulva was cleaned and disinfected, the cervix was opened and the fallopian tubes were flushed. The embryo transfer catheter was inserted deep into one uterine horn using the rectal palpation method. The plunger of a syringe connected to embryos transfer catheter was pushed to inject the embryos that had developed to the blastocyst stage after in vitro fertilization, and the embryo transfer catheter was then withdrawn. The pregnancy status was assessed by B-mode ultrasonography on days 45-50 of pregnancy, and the pregnancy rate was calculated.

### 2. Experimental Results

As can be seen from Table 3, compared with the control group (68.2±2.5%), treatment groups 1/2/4/5/6/7 did not affect the 48-h cleavage rate of the in vitro fertilized embryos, but all enhanced the blastocyst development efficiency; the combined treatments VitC+L-carnitine, VitC+THPO, L-carnitine+THPO, and VitC+L-carnitine+THPO produced synergistic effects and all achieved significantly higher blastocyst development rates than the VitC-only, L-carnitine-only, and THPO-only treatment groups.

As can be seen from Table 4, compared with the control group (40.0%), the administration of L-carnitine alone can improve the pregnancy rate after embryo transfer, but the combined treatments VitC+L-carnitine, VitC+THPO, L-carnitine+THPO, and VitC+L-carnitine+THPO all achieved significantly higher pregnancy rates after embryo transfer than the VitC-only, L-carnitine-only, and THPO-only treatment groups.

Therefore, the combined administration of two or three of VitC, L-carnitine, and THPO can significantly improve the development efficiency and quality of bovine in vitro fertilized embryos.

TABLE 3
The effect of the combined administration of VitC,
L-carnitine, and THPO during in vitro maturation
of bovine oocytes on the embryo development rate.

| | Number of | | D8 blastocyst |
|---|---|---|---|
| | oocyte | 48-h cleavage rate (%) | rate (%) |
| Control group | 201 | 68.2 ± 2.5 | 26.8 ± 2.2^{a} |
| Treatment group 1 | 198 | 68.7 ± 3.1 | 33.1 ± 1.3^{b} |
| Treatment group 2 | 188 | 69.5 ± 4.3 | 32.4 ± 0.8^{b} |
| Treatment group 3 | 192 | 67.4 ± 3.8 | 32.2 ± 2.0^{b} |
| Treatment group 4 | 187 | 72.1 ± 2.9 | 37.5 ± 1.2^{c} |
| Treatment group 5 | 205 | 67.9 ± 4.8 | 36.9 ± 1.0^{c} |
| Treatment group 6 | 210 | 68.2 ± 3.3 | 35.9 ± 0.8^{c} |
| Treatment group 7 | 224 | 70.5 ± 3.2 | 38.3 ± 1.5^{c} |

Note:
The superscripts ^{a}, ^{b}, and ^{c} indicate significant differences, and the significance is tested using one-way analysis of variance. Cleavage rate = number of two-cell embryos/number of oocytes × 100%, blastocyst rate = number of blastocysts/number of oocytes × 100%.

TABLE 4
The effect of the combined administration of VitC, L-carnitine,
and THPO during the in vitro maturation of bovine oocytes
on the pregnancy rate after embryos transfer.

| | Number of | Number of | |
|---|---|---|---|
| | recipients | pregnancies | Pregnancy rate (%) |
| Control group | 25 | 10 | 40.0%^{a} |
| Treatment group 1 | 26 | 11 | 42.3%^{a} |
| Treatment group 2 | 26 | 12 | 46.2%^{b} |
| Treatment group 3 | 26 | 11 | 42.3%^{a} |
| Treatment group 4 | 28 | 15 | 53.5%^{c} |
| Treatment group 5 | 25 | 13 | 52.0%^{c} |
| Treatment group 6 | 27 | 14 | 51.9%^{c} |
| Treatment group 7 | 30 | 17 | 56.7%^{c} |

Note:
The superscripts ^{a}, ^{b}, and ^{c} indicate significant differences, and the significance is analyzed using chi-square tests. Pregnancy rate = number of pregnant female animals/number of recipient female animals × 100%.

Although the present disclosure has been described with reference to exemplary embodiments, it should be understood that the present disclosure is not limited to the disclosed exemplary embodiments. Various modifications and variations can be made to the exemplary embodiments of the present specification without departing from the scope or spirit of the present invention. The scope of the claims should be based on the broadest interpretation to encompass all modifications and equivalent structures and functions.

1. A composition for improving a quality of in vitro maturation of mammalian oocytes and a pregnancy rate of recipient mammalians after in vitro fertilization of the in vitro matured oocytes and embryo transfer, the composition comprising:
(a) Vitamin C or a precursor thereof and thrombopoietin (THPO);
(b) L-carnitine and THPO; or
(c) Vitamin C or a precursor thereof, L-carnitine, and THPO.

2. The composition for improving the quality of in vitro maturation of mammalian oocytes and the pregnancy rate of recipient mammalians after in vitro fertilization of the in vitro matured oocytes and embryo transfer according to claim 1, wherein:
a weight ratio of the vitamin C or its precursor to the THPO in (a) is 200-1000:1;
a weight ratio of the L-carnitine to THPO in (b) is 500-3000:1; and
a weight ratio of the vitamin C or its precursor, the L-carnitine to the THPO in (c) is (200-1000): (500-3000):1.

3. The composition for improving the quality in vitro maturation of mammalian oocytes and the pregnancy rate of recipient mammalians after in vitro fertilization of the in vitro matured oocytes and embryo transfer according to claim 1, wherein an amount of vitamin C or its precursor is able to make a working concentration of vitamin C reach 0.5-500 μg/ml during in vitro culture; an amount of L-carnitine is able to make a working concentration of L-carnitine reach 10-1000 μg/ml during in vitro culture; or an amount of THPO is able to make a working concentration of THPO reach 0.001-5 μg/ml during in vitro culture.

4. The composition for improving the quality of in vitro maturation of mammalian oocytes and the pregnancy rate of recipient mammalians after in vitro fertilization of the in vitro matured oocytes and embryo transfer according to claim 1, wherein the composition further comprises a basal culture medium.

5. A method for improving the quality of in vitro maturation of mammalian oocytes and the pregnancy rate of recipient mammalians after in vitro fertilization of the in vitro matured oocytes and embryo transfer, the method comprising a step of contacting the embryo with a composition according to claim 1 in vitro.

6. The method for improving quality of in vitro maturation of mammalian oocytes and the pregnancy rate of recipient mammalians after in vitro fertilization of the in vitro matured oocytes and embryo transfer according to claim 5, wherein the improving comprises improving the efficiency and/or quality of embryo development.

7. A method for improving in vitro maturation of mammalian oocytes, the method comprising a step of contacting the oocytes with the composition according to claim 1 in vitro.

8. The method for improving in vitro maturation of mammalian oocytes according to claim 7, wherein the improvement comprises increasing a blastocyst development rate, a number of blastocyst cells, reducing a number of blastocyst apoptotic cells, and increasing the pregnancy rate after embryo transfer.

9. Use of the composition according to claim 1 in animal genetic improvement, elite breed propagation, and in vitro embryo production or culture.
